(19) United States
(12) Patent Application Publication
(10) Pub. No.: US 2025/0318913 A1
(43) Pub. Date: Oct. 16, 2025

(54) MARKER INTEGRALLY FORMED WITH ORAL STENT

(71) Applicant: NEOBIOTECH CO., LTD, Gangwon-do (KR)

(72) Inventor: CHAE HEON HEO, Seoul (KR)

(21) Appl. No.: 18/868,980

(22) PCT Filed: Dec. 30, 2022
(86) PCT No.: PCT/KR2022/021731

§ 371 Date: Nov. 25, 2024

(30) Foreign Application Priority Data
May 27, 2022 (KR) 10-2022-0065369

(51) Int. Cl. A61C 8/00; A61B 90/00

(52) U.S. Cl. CPC A61C 8/0089; A61B 90/39; A61B 2090/3966; A61B 2090/3979

(57) ABSTRACT

A marker integrally formed with an oral stent includes a stent located in the oral cavity of a patient and capable of undergoing IR tracking and CBCT imaging and a marker part connected to one side of the stent and exposed to the outside of the oral cavity when the stent is placed in the oral cavity to track the coordinates of the stent. The marker part may have a fixed joint integrally connected to a side surface of the stent, a movable joint that rotates left and right at the fixed joint to adjust the angle of the marker part, and a gear wheel formed at each of the upper end of the fixed joint and the lower end of the movable joint. According to the coupling position of the gear wheel, the angle of the marker part is adjusted and fixed.

### BACKGROUND

### 1. Technical Field

The present invention relates to a marker integrally formed with an oral stent, and more specifically, to a marker integrally formed with an oral stent, in which the position of a stent fixed in an oral cavity of a patient is recognized by an infrared (IR) camera of a marker part located outside the oral cavity so that the coordinates of the stent can be tracked in real time.

### 2. Background Art

Implant procedures for replacing teeth with implants includes a process of making a gum incision to expose an alveolar bone and, then forming a hole in the alveolar bone into which a fixture is implanted using a drill. Assistant tools referred to as stents are used to guide an implantation direction of the fixture and a location of the hole that is formed in the alveolar bone by a surgeon during drilling work of forming the hole.

EP 2 236 104 B1 discloses a method and device for medical navigation in which a positional relationship between an instrument and a part of a patient's body is ascertained. This publication enables accurate surgical navigation to be performed in a simple and reliable manner.

WO 2013/052187 A2 discloses an on-tool tracking device for providing guidance by performing tracking during computer-assisted surgery using a surgical instrument. The on-tool tracking device includes a housing that has a pair of cameras. A visible range of cameras can be physically or electronically adjusted depending on a desired visible range required for a specific computer-assisted surgical procedure performed by the on-tool tracking device.

US 2012/0015329 A1 discloses a dental tool including a first optically-visible marker that is coupled thereto. A second optically-visible marker is attached to a structure such as a tooth inside a subject's mouth. Two or more tool-cameras are coupled to the tool at a fixed position.

WO 2006/131373 A2 discloses a device for contactless determination and measurement of a spatial position and/or spatial orientation of bodies using a tracking system, by means of which the bodies are located and brought into relations with one another, the tracking system, or at least components or modules thereof, being mobile.

DE 20 2011 005 573 U1 discloses a device for fixing a human body or a part of the human body, particularly for attaching a medical component, a marker, or a surgical instrument thereto. The device includes a casting element that can be positioned on a surface of the human body. The casting element has at least one adhesive layer.

### SUMMARY

An object of the present invention is to provide a marker integrally formed with an oral stent, in which the position of a stent fixed in an oral cavity of a patient is recognized by an IR camera of a marker part located outside the oral cavity so that the coordinates of the stent can be tracked in real time.

Another object of the present invention is to provide a marker integrally formed with an oral stent enabling an angle of a movable joint to be adjusted in a certain range by gear wheels after the movable joint is teeth-coupled to a fixed joint, and enabling a fixed state to be maintained after the angle is adjusted.

Still another object of the present invention is to provide a marker integrally formed with an oral stent enabling fine and accurate angle adjustment with a reference line and an angle adjustment scale when an angle of a movable joint is adjusted to left or right at a fixed joint.

Still another object of the present invention is to provide a marker integrally formed with an oral stent in which radiopaque markers are formed at the upper end of a fixed joint, the upper end of a movable joint, and a side surface of the stent, respectively, so that the radiopaque markers can be recognized during cone beam computed tomography (CBCT) imaging.

According to the present invention, there is provided, as a means for achieving the above-described objects, a marker integrally formed with an oral stent that is basically characterized to be technically configured to include: a stent that is located in the oral cavity of a patient and capable of undergoing IR tracking and CBCT imaging; and a marker part that is connected to one side of the stent and is exposed to the outside of the oral cavity when the stent is placed in the oral cavity of the patient to track the coordinates of the stent,
wherein the marker part has a fixed joint that is integrally connected to a side surface of the stent, and a movable joint that rotates left and right at the fixed joint to adjust an angle of the marker part, in which a gear wheel is formed at the upper end of the fixed joint and the lower end of the movable joint, respectively, and the angle of the marker part is adjusted and fixed according to the coupling position of the gear wheel,
a part at which the fixed joint and the movable joint are connected is selected, a reference line for determining the angle is formed at the selected part of the upper end of the fixed joint, and an angle adjustment scale is formed at the lower end of the movable joint to enable a rotation angle of the movable joint to be finely adjusted,
multiple IR markers are formed at tips of the movable joint of the marker part, and coordinates of the IR markers are recognized in real time with an IR camera, and
radiopaque markers are formed at the upper ends of the fixed joint and the movable joint of the marker part and a side surface of the stent, respectively, so that the radiopaque markers are recognizable during CBCT imaging.

As described above, according to the present invention, a stent located in the oral cavity and a marker part located outside of the oral cavity are integrally formed with each other. Hence, since an IR marker of the marker part located outside of the oral cavity of the patient is recognized as a location of the stent fixed in the oral cavity with an IR camera, coordinates of the stent located in the oral cavity can be tracked in real time.

In addition, a fixed joint and a movable joint are teeth-coupled by gear wheels. Hence, an angle of the movable joint is adjusted in a certain range by the gear wheels, and a fixed state can be maintained after the angle is adjusted.

In addition, according to the present invention, since the angle can be adjusted with a reference line and an angle adjustment scale when the angle of the movable joint is adjusted to left or right at the fixed joint, fine and accurate angle adjustment is performed.

In addition, since radiopaque markers are formed at the upper ends of the fixed joint and the movable joint and a side surface of the stent, respectively and the radiopaque markers can be recognized during CBCT imaging, an effect of recording and storing of the markers in CBCT data is achieved.

### BRIEF DESCRIPTION OF THE DRAWINGS

FIG. 1 is a perspective view of a marker integrally formed with an oral stent according to the present invention.

FIG. 2 is a perspective view of the stent and a fixed joint according to the present invention.

FIG. 3 is a partial perspective view of the fixed joint and a movable joint according to the present invention.

FIG. 4 is a plan view illustrating a reference line and an angle adjustment scale according to the present invention.

### DETAILED DESCRIPTION

Hereinafter, an example of the present invention will be described in detail with reference to the accompanying drawings. However, the present invention is not limited to the example described herein and can be implemented as various other embodiments. In addition, a part unrelated to the description is omitted from the drawings in order to clearly describe the present invention.

In the entire specification, when a certain part “comprises” a certain configurational element, this means that another configurational element is not excluded but the other configurational element can be further included unless specifically described otherwise.

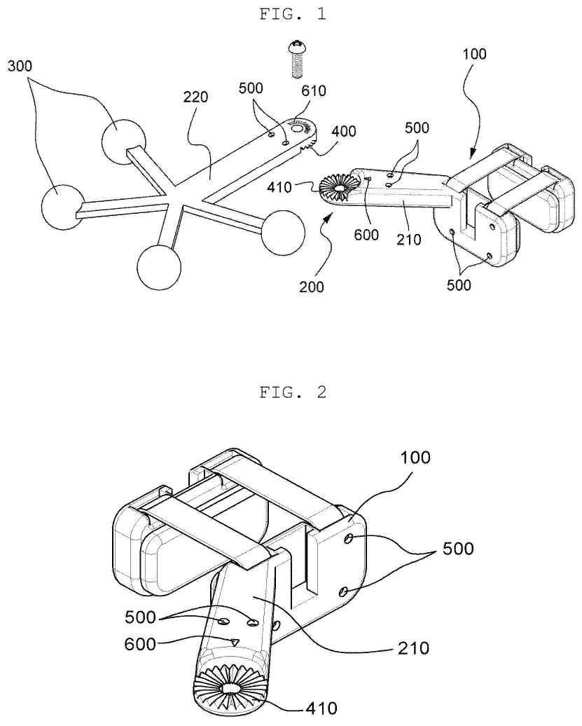

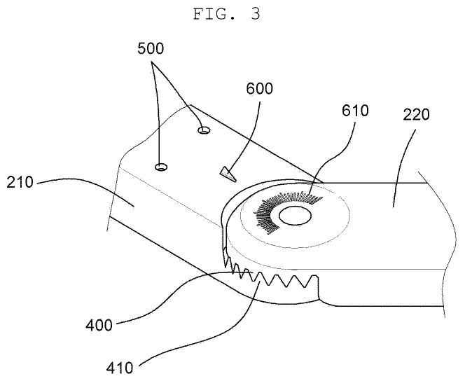

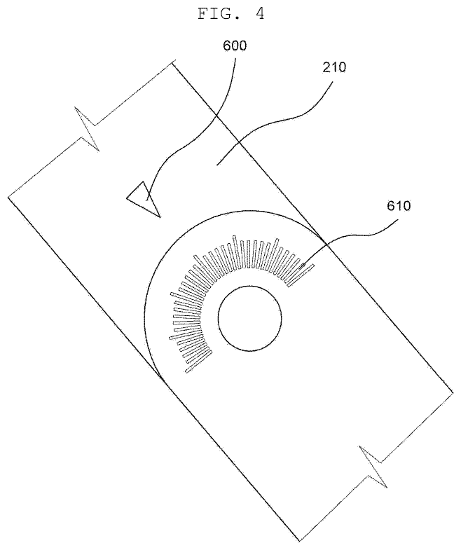

FIG. 1 is a perspective view of a marker integrally formed with an oral stent according to the present invention, FIG. 2 is a perspective view of the stent and a fixed joint according to the present invention, FIG. 3 is a partial perspective view of the fixed joint and a movable joint according to the present invention, and FIG. 4 is a plan view illustrating a reference line and an angle adjustment scale according to the present invention.

As depicted in FIGS. 1 to 4, the present invention is configured to include a stent 100 that is located in the oral cavity of a patient and capable of undergoing IR tracking and CBCT imaging; and a marker part 200 that is connected to one side of the stent 100 and is exposed to the outside of the oral cavity when the stent 100 is placed in the oral cavity of the patient to track the coordinates of the stent 100.

The stent 100 is used in dentistry and fulfills a function of guiding to an implanting location in order to determine the implanting location of an implant. The stent 100 used in the present invention has both radiopaque markers 500 that can appear on a CT image and IR markers 300 that can be recognized by an IR camera, simultaneously.

Hence, it is possible to merge CBCT data and a design file of the stent 100 on 3D software, and it is possible to track an actual location of the stent 100 in real time.

A usage process of the IR markers 300 and the radiopaque markers 500 can be briefly described as follows.
CBCT imaging: When the stent 100 according to the present invention is placed in the oral cavity of the patient and CBCT imaging is performed, the radiopaque markers 500 formed on the stent 100 are recognized during the CBCT imaging.
A patient's data and 3D data of the stent 100 are merged by matching the radiopaque markers 500 in CBCT data with markers in an STL design file of the stent 100 stored on the 3D software. Implant placement planning information is designed with the merged data by using 3D Planning CAD software.
IR camera (not shown): The IR camera is separately installed externally and recognizes locations of the IR markers 300.
IR marker: When the stent 100 is placed in the oral cavity of the patient, the stent 100 is located in the oral cavity, and the IR markers 300 are located outside the oral cavity. In this case, the IR camera recognizes the locations of the IR markers 300 and tracks coordinates of the stent 100 in real time.
Then, the coordinates of the stent 100 obtained by the IR markers 300 and the IR camera are transmitted to 3D Visualization software, and a location of the patient are displayed on the 3D Visualization software in real-time.

In design to achieve this process, multiple IR markers 300 are formed at tips of the movable joint 220 of the marker part 200, and coordinates of the IR markers 300 can be recognized in real time by the IR camera. In addition, in the design, the radiopaque markers 500 are formed at the upper ends of the fixed joint 210 and the movable joint 220 of the marker part 200 and a side surface of the stent 100, respectively, so that the radiopaque markers 500 are recognized during CBCT imaging.

The marker part 200 includes the fixed joint 210 that is integrally connected to a side surface of the stent 100 and the movable joint 220 that rotates left and right at the fixed joint 210 to adjust an angle of the marker part 200. Gear wheels 400 and 410 are formed at the upper end of the fixed joint 210 and at the lower end of the movable joint 220, respectively, and the angle of the marker part 200 is adjusted and fixed according to the coupling positions of the gear wheels 400 and 410. After the angle is fixed, a fixed state is maintained. In this angle adjustment configuration, angle adjustment needs to be performed depending on impression acquisition locations of the stent 100.

Here, in the drawings, a configuration is described as an example in which the marker part 200 can separate the fixed joint 210 and the movable joint 220 from each other to adjust the angle thereof. However, the present invention is not limited thereto, and it is noted that, in a case where there is no need to adjust the angle, the marker part 200 can be formed as a single body without separating the fixed joint and the movable joint from each other.

On the other hand, a part at which the fixed joint 210 and the movable joint 220 are connected is selected, a reference line 600 for determining the angle is formed at the selected part of the upper end of the fixed joint 210, and an angle adjustment scale 610 is formed at the upper end of the movable joint 610 to enable a rotation angle of the movable joint 220 to be finely adjusted. The reference line 600 and the angle adjustment scale 610 enable accurate angel adjustment to be performed.

The necessity of the reference line 600 and the angle adjustment scale 610 is as follows.

Dental treatment and surgery are not performed immediately after CBCT imaging in some cases, and depending on convenience, the stent 100 and the part of the radiopaque markers 500 need to be separated from each other. In these cases, when the stent 100 and the radiopaque markers 500 are placed at the exact locations (angles) imaged in the CBCT data, the patient's location can be accurately identified during surgery. However, when the stent and the radiopaque markers are placed at different locations (angles), coordinate values for treatment and surgery may be changed, and a medical accident may be potentially caused. In this case, the reference line 600 and the angle adjustment scale 610 can fulfill functions thereof in the above-described case.

As described above, the description provided above focuses on the examples; however, the example is described only as an example, and the present invention is not limited thereto. In addition, it is possible for those of ordinary skill in the art to which the present invention pertains to find that various modifications and applications of the present invention which are not exemplified in the description provided above can be achieved within the scope not departing from intrinsic characteristics of the example. For example, each configurational element which is specifically described in the examples can be modified to be realized. Further, differences related to such modifications and applications are to be construed to be included in the scope of the present invention which is specified in the accompanying claims.

1. A marker integrally formed with an oral stent comprising:
a stent configured to be located in an oral cavity of a patient and capable of undergoing infrared (IR) tracking and cone beam computed tomography (CBCT) imaging; and
a marker part connected to one side of the stent and configured to be exposed to the outside of the oral cavity when the stent if placed in the oral cavity of the patient to track the coordinates of the stent, the marker part comprising:
a fixed joint integrally connected to the stent;
a movable joint configured to rotate left and right at the fixed joint to adjust an angle of the marker part; and
gear wheels formed at an upper end of the fixed joint and at a lower end of the movable joint, respectively, wherein the angle of the marker part is adjusted and fixed according to coupling positions of the gear wheels,
wherein a part at which the fixed joint and the movable joint are connected is selected, a reference line for determining the angle is formed at the selected part of the upper end of the fixed joint, and an angle adjustment scale is formed at an upper end of the movable joint to enable a rotation angle of the movable joint to be finely adjusted.

2. The marker integrally formed with an oral stent according to claim 1, wherein multiple IR markers are formed at tips of the movable joint of the marker part, and coordinates of the IR markers are recognized in real time with an IR camera.

3. The marker integrally formed with an oral stent according to claim 1, wherein radiopaque markers are formed on the upper ends of the fixed joint and the movable joint of the marker part and a side surface of the stent, respectively, so that the radiopaque markers are recognizable during CBCT imaging.
